(19) United States
(12) Patent Application Publication
(10) Pub. No.: US 2008/0138821 A1
(43) Pub. Date: Jun. 12, 2008

(54) Process for prognosis of disease using gene GNA11

(76) Inventors: Katrin Riemann, Essen (DE); Winfried Siffert, Gelsenkirchen (DE)

Correspondence Address: AMSTER, ROTHSTEIN & EBENSTEIN LLP, 90 PARK AVENUE, NEW YORK, NY, 10016, US

(21) Appl. No.: 11/983,449

(22) Filed: Nov. 9, 2007

(30) Foreign Application Priority Data
Nov. 10, 2006 (DE) 10 2006 053 138.8

(51) Int. Cl. C12Q 1/68

(52) U.S. Cl. 435/6

(57) ABSTRACT

The invention relates to the use of a genomic gene modification in the gene for the Gα₁₁ subunit of human G-proteins which sub-unit is encoded by the gene GA11, for the prognosis of disease risks, disease developments and the response to disease therapies by pharmacological and non-pharmacological measures and for predicting undesired drug effects. The invention relates moreover to the provision of individual gene modifications and haplotypes by means of which further gene modifications suitable for use for the above-mentioned purposes can be detected and validated. Such gene modifications may consists of a G(−659)C polymorphism being detected in the promoter of the gene. The gene modifications can be detected individually or in any desired combination by means of processes familiar to the expert.

### CROSS-REFERENCE TO RELATED APPLICATION

This application claims the benefit of German Patent Application No. 10 2006 053 138.8, filed on Nov. 10, 2006, the content of which is herein incorporated by reference.

### FIELD OF THE INVENTION

The invention relates to an in vitro process in which a gene modification is used for diagnostic purposes, in particular for the prognosis of disease risks, disease developments and means for finding drug targets.

### BACKGROUND OF THE INVENTION

All cells of the human body possess membrane receptors at their surfaces via which all cell functions are controlled. Such receptors include the so-called heptahelical receptors for hormones, neurotransmitters and chemokines. In addition, there are numerous receptors for growth factors and receptors with an intrinsic tyrosine kinase activity such as receptors for insulin, insulin-like growth factor, epidermal growth factor, platelet-derived growth factor and many more. In addition, there are many receptors which are responsible for the regulation of blood formation such as the receptor for erythropoietin. Cell growth, motility, gene expression, apoptosis and chemotaxis, among others, are controlled via such receptors. The above-mentioned receptors transmit their signals into the cell interior via the activation of so-called heterotrimeric G-proteins. These G-proteins consist of a large family of different isoforms and are composed of different α, β and γ-subunits respectively. At present, 5 β-subunits, 13 γ-subunits and more than 20 α-subunits are known which are coded by different genes (Farfel Z et al., The expanding spectrum of G-protein diseases. N Engl J. Med. 1999 Apr. 1; 340(13):1012-20). By way of the combination of these different α, β and γ-subunits, numerous different heterotrimeric G-proteins are formed.

In this connection, the isoform combination determines which heterotrimer can be activated by a specific receptor. From the functional point of view, the βγ-subunits must be considered as a monomer. At rest, the α-subunit includes GDP in the bound state (FIG. 1). Following activation of a coupled receptor, the α-subunit liberates GDP in exchange for GTP and the dissociation of the βγ-subunits from the α-subunits takes place. Both the free α-subunits and the βγ-subunits can control the activity of numerous different effectors. These include, for example, ion channels, the adenylyl cyclases, the PI3 kinase, different MAP inases. The α-subunits dispose of an intrinsic GTPase activity which hydrolyses the GTP bound following activation to GDP. Subsequently, the liberated βγ-subunits reassociate with the α-subunit as a result of which the activation cycle is terminated. As a result, the heterotrimer is available for a fresh activation cycle (Bourne H R. How receptors talk to trimeric G-proteins. Curr Opin Cell Biol. 1997; 9(2):134-42).

The Gα₁₁ subunit is expressed in all body cells of humans. Its stimulation leads to the activation phospholipase C, for example, and consequently to an increase in the intracellular Ca²⁺ concentration. In this way, Ca²⁺-dependent processes, for example, can be activated. Moreover, Gα₁₁ is able to regulate the activity of ion channels, e.g. of potassium or calcium channels. Almost all known receptors couple to Gα₁₁, e.g. the receptors for adenosine, adrenalin, angiotensin, bradykinin, calcitonin, dopamine, endothelin, histamine, noradrenaline, P2-purinergic receptors, thrombin, thromboxane, vasopressin and many more. Following the stimulation of Gα₁₁-coupled receptors, an apoptosis is induced in many cell types such that a connection with tumour diseases and their development and therapy responses, but also a connection with inflammatory and immunological diseases and their development and therapy responses is obtained. In addition, diverse metabolic pathways are regulated by Gα₁₁. It is of great medical relevance to define prognosis factors for the development of cancer diseases which provide information on the response to certain forms of therapy or which are predictive of the occurrence of metastases, tumour progression and survival. So far, prognosis factors generally known to the expert are used in the field of medicine. These include, for example, the size of the tumour, its depth of penetration into the surrounding tissue, organ exceeding growth, penetration into the blood or lymphatic vessels or lymph nodes and the degree of differentiation of the tumour cells. In addition, there are some relatively non-specific serological markers. The process for classifying tumours is generally referred to as “staging” and “grading”. In general, the rule applies that the presence of distant metastases and a lower degree of differentiation represent parameters which are highly unfavourable from the prognosis point of view. Nevertheless, general experience in the field of medicine has shown that patients at the same tumour stage exhibit dramatically different disease developments. Whereas, in the case of some patients, a rapid progression of the disease and the occurrence of metastases and relapses are observed, the disease comes to a standstill for obscure reasons in the case of other patients. Metastasis may occur locally, regionally and far from the parent tumour. To this end, it is necessary for a large number of tumour cells to reach the adjacent tissue via the lymph or blood pathway by simple swimming in fluid or by contact. A tendency towards relapse should be understood to mean the renewed occurrence of a tumour following the incomplete or partial surgical removal of the tumour. In this case, it is not a renewed malignant transformation which is involved, but the regrowth of an incompletely removed tumour tissue. A recrudescence is also possible by metastases which may remain latent for many years. Progression should be understood to mean the reoccurrence of a tumour with a higher grading (greater dedifferentiation) or the renewed occurrence of metastases.

Quite obviously, there are numerous individual, non-recognised biological variables which determine to a large extent the course a tumour disease takes independently of staging and grading. Such factors include genetic host factors. In addition, it is desirable to develop genetic markers which are predictive of the occurrence of tumours. Such markers fulfil the task of ensuring that the individuals affected are submitted to further screening measures (serology, x-rays, ultrasound, NMR) at an early stage. In this way, cancer diseases can be recognised and therapeutically tackles in the early stages as a result of which the chances of healing and survival are markedly better in the case of tumours in the early stage than in the case of tumours which have progressed.

### SUMMARY OF THE INVENTION

It is the task of the invention,

(a) to provide function-modifying genomic polymorphisms and haplotypes in the gene GNA11 which lead either to an amino acid exchange, or
(b) lead to a change in the protein expression or
(c) which are suitable for finding and/or validating further polymorphisms or haplotypes in the gene GNA11;
(d) to provide nucleotide exchanges and haplotypes which are suitable for predicting the general disease risks and developments;
(e) to provide nucleotide exchanges and haplotypes which are suitable for predicting the general response to drugs and the side effects;
(f) to provide nucleotide exchanges and haplotypes which are suitable for predicting in general the effect of other forms of therapy such as radiation, warmth, heat, cold, motion.

These objects are achieved by an in-vitro process in which, in order to predict disease risks, disease developments, drug risks and to find drug targets, a search for a gene modification in the promoter region of the gene GNA11 and/or intron 1 of the gene GNA11 of the human chromosome 19p13.3 is carried out.

### BRIEF DESCRIPTION OF THE DRAWINGS

FIG. 1 shows the Gα11 signal pathway. The diagram shows the way in which the Gα11 pathway is connected, following receptor stimulation, with diverse signal transduction components, including ion channels, transcription factors and the synthesis of eicosanodia. PLC, phospholipase C; IP3, inositol trisphophate; PKC, protein kinase C; PLA2, phospholipase A2; AA, arachidonic acid; MLCK, myosin light chain kinase; CaM, calmoduline; p42/p44-MAP-kinase; Kontraktion—contraction; Wachstum—growth.

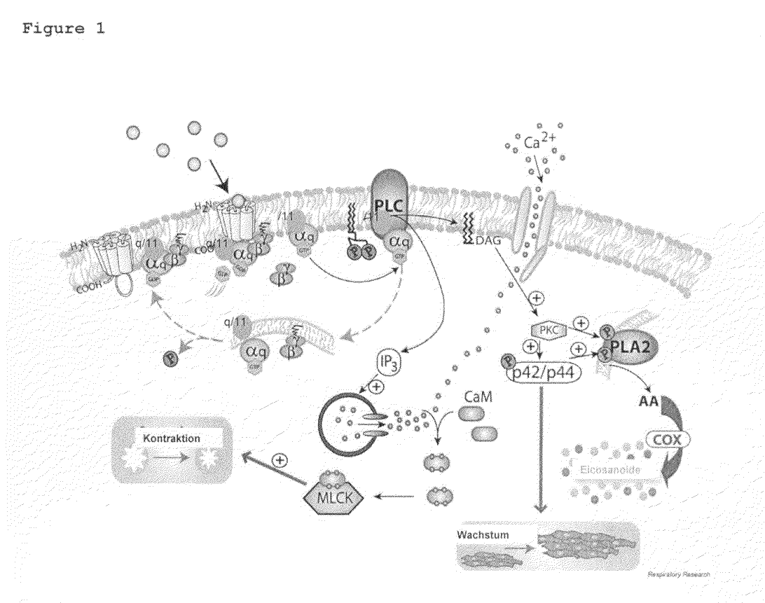

FIG. 2 shows the intron/exon structure of human GNA11 and the position of the G(−659)C promoter polymorphism as well as the intron I polymorphisms G(1606)T and C(10564)T (not according to scale).

FIG. 3 shows a haplotype analysis of the promoter polymorphism G(−659)C (rs11084997) with the intronI-polymorphisms G(1606)T and C(10564)T (rs3968546).

FIG. 4 shows the results of the electrophoretic mobility shift assays (EMSA) with the constructs containing the (−659)GG or the CC genotype in the promoter of GNA11. After adding cell nucleus extract, an increased binding of nucleus protein to the “CC construct” is recognised which exhibits a further binding site for the transcription factor AP2-alpha.

FIG. 5A-5B shows the genotype-dependent activity of the GNA11 promoter. The promoter construct −849/−274 with the GG genotype or the CC genotype was transfected into HEK cells and HELA cells. The secretion of alkaline phosphatase was measured after different time periods. A significantly increased activity of the promoter can be seen with the GG genotype both in the case of HELA-cells (A: in this case 12 h after transfection) as well as in HEK cells (B: in this case 8 h after transfection).

FIG. 6 shows the expression of Gα11 mRNA in bladder tumor tissue as a function of the G(−659)C polymorphism. The quotient Gα11/β-actin mRNA is shown.

FIG. 7A-7B shows the GNA11 G(−659)C polymorphism and the cyclic parameter for patients. The pulmonary arterial mean pressure (A) is shown as a function of the genotype and the heterozygous GC genotypes are taken together with the homozygous CC genotypes (B). Y-axis: Pulmonary arterial mean pressure (mmHg).

FIG. 8A-8B shows the GNA11 G(−659)C polymorphism and cyclic parameter in the case of patients before the operation. The pulmonary vascular resistance (A) is shown as a function of the genotype and the heterozygous GC genotypes as taken together with the homozygous CC genotypes (B).

FIG. 9A-9B shows the GNA11 G(−659)C polymorphism and the echocardiography in the case of persons with coronary heart disease. The ejection fraction is shown in % in the case of all persons examined before the operation (A) and persons with a myocardial infarction (B) as a function of the genotype.

FIG. 10A-10C shows the changes in weight over time in treatment with a placebo or sibutramine for the GNA11 (−659) GG genotype (A), the GC genotype (B) and the CC genotype (C). It can be seen that only the CC genotype benefits from a therapy with 15 mg of sibutramine daily, whereas the GG-/GC genotypes may lose weight without a drug. Y-axis: Gewichtsveranderung—change in weight; X-axis: Woche=week.

FIG. 11A-11B shows the use of a gene modification in the human GNA11 gene for predicting an increase in the heart rate during therapy with sibutramine. The effects are shown as a function of the genotype of the G(−659)C polymorphism. Y-axis: Change in heart rate (bpm); X-axis: Woche=week.

FIG. 12A-12B shows the use of a gene modification in the human GNA11 gene for predicting an increase in the diastolic blood pressure during therapy with sibutramine. The time-dependent changes in the diastolic blood pressure (DBP) over time are shown as a function of the genotype of the G(−659)C polymorphism. Y-axis: Diastolic blood pressure (DBP) changes (mmHg); X-axis: Woche=week.

FIG. 13A-13B shows the use of a gene modification in the human GNA11 gene for predicting an increase in the systolic blood pressure during therapy with sibutramine. The time-dependent changes in the systolic blood pressure (SBP) over time are shown as a function of the genotype of the G(−659)C polymorphism. Y-axis: Systolic blood pressure (SBP) changes (mmHg); X-axis: Woche=week.

FIG. 14A-14C shows a GNA11 intron I polymorphism C(10564)T and the time up to metastasis (A), up to progression (B) and up to the formation of a recrudescence (C) in the case of patients with bladder carcinoma. Y-axis in A: % ohne Metastasen—% without metastases; Y-axis in B: % ohne Progression—% without progression; Y-axis in C: % ohne Rezidiv—% without recrudescence; X-axis in A-C: Zeit (Monate)—time (months).

FIG. 15 shows a GNA11 C(10564)T polymorphism and the survival in the case of patients with a bladder carcinoma. Y-axis: % survival; X-axis: time(months).

### DETAILED DESCRIPTION OF THE INVENTION

The human gene GNA11, which codes for Gall, is positioned on chromosome 19p13.3 (Accession no. NW 927173 of the gene bank of the National Centre for Biotechnology Information (NCBI)).

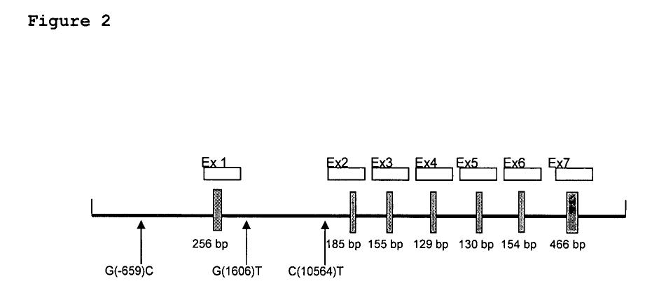

Suitable gene modifications for carrying out the process according to the invention are those positioned in front of exon 1 in the promoter of the gene, such as the gene-polymorphism G(−659)C, rs11084997 (FIG. 2) which has not yet been described in the literature as being connected with specific diseases. The promoter sequence is modified by the polymorphism in this section of GCTGCCC to GCTCCCC. Two further gene polymorphisms suitable according to the invention are also situated in the GNA11 promoter region at A(−761)C (rs7245431) and at G(−626)A (rs11084998). The numbering of these SNPs is effected in such a way that the number +1 is allocated to the nucleotide A of the start codon ATG. Since, according to convention, the number 0 does not exist, the nucleotide situated in front of A of start codon ATG has been allocated the number −1.

Moreover, gene modifications suitable for carrying out the process according to the invention are those in intron 1 of the human GNA11 gene, and thus the G(1606)T polymorphism such that a partial sequence of intron 1 is modified from AAGGGTT to AAGTGTT. In addition, a C(10564)T polymorphism (rs 3968546) has been detected in intron 1 such that a partial sequence of intron 1 is modified from GGCCGGA to GGCTGGA. The numbering of these two SNPs is carried out in such a way that the number +1 is allocated to the nucleotide A of the start codon ATG and counting is carried out consecutively beyond the exon into the intron region.

The polymorphisms found according to the invention in gene GNA11 are in reciprocal coupling imbalance and can be used as such or in any desired combination for predicting disease developments or the effects of drugs. The invention consequently relates, on the one hand, to the detection of the gene modifications and of haplotypes or coupling imbalances. The invention relates on the other hand to the use of the gene modifications determined for predicting disease risks and disease developments. The basis for this is the modified activatability of Gall as a result of the gene modifications described. These then determine simultaneously a different response to drugs dependent on the gene status.

The process according to the invention using the SNPs indicated above can be carried out by means of any desired process familiar to the expert. Such processes are, for example, direct sequencing, PCR with subsequent restriction analysis, reverse hybridisation, dot-blot or slot-blot processes, mass spectrometry, Taqman® technology or Light-Cycler® technology, Pyrosequencing®, Invader® technology, Luminex processes. In addition, these gene polymorphisms can simultaneously be detected on a DNA chip following multiplex PCR and hybridisation.

It has been found that modifying the gene expression Gα₁₁ (overexpression or lacking expression) in the animal experiment or at cellular level leads to a series of disease states or phenotypes such as

### 1. The overexpression of Gαq/11 in the heart leads to hypertrophy, heart failure and apoptosis.

### 2. Constitutively active Gαq/11 inhibits cell proliferation and induces apoptosis via the protein kinase C pathway.

### 3. Knockout of Gα11 shows neither behavioural disturbances nor morphological changes in mice, the animals live and are fertile.

4. On the other hand, knockout of Gαq/11 brings about considerable defects; the embryos die as a result malformation of the heart. If a copy of the gene concerned is present, the mice die shortly after birth as a result of the malformation of the skull or the heart.

### 5. Gα11 participates in the signal transduction of insulin.

6. A reduced expression of Gα11 and MCT1 is associated with the occurrence of breast cancer (Asada K et al. Reduced expression of GNA11 and silencing of MCT1 in human breast cancers. Oncology 2003; 64:380-388).

These few examples alone prove that function-modifying mutations in Gα11 or the underexpression or overexpression of the proteins can lead to different diseases and/or malfunctions also in man.

The distribution of the promoter −G(−659)C, intron 1-G(1606)T and intron1-C(10564)T polymorphisms in different ethnic groups and the use of this genotype for finding further relevant polymorphisms and haplotypes. For this purpose, different DNA samples from Caucasians, black Africans and Chinese were genotyped. The result is shown in the following tables.

TABLE 1

| | White | | Black |
|---|---|---|---|
| G(-659)C | Caucasians | Chinese | Africans |
| n % | 95 | 93 | 94 |
| GG | 39 (41%) | 56 (60.2%) | 59 (62.7%) |
| GC | 48 (50.5%) | 35 (73.6%) | 31 (33%) |
| CC | 8 (8.5%) | 2 (2.2%) | 4 (4.3%) |
| % G | 66.3 | 79.0 | 79.3 |

This genotype distribution is significantly different in the Chi² test with Chi of 13.8 and a p<0.008. The GG geno-type occurs most frequently in black Africans.

TABLE 2

| Intron 1 | White | | Black |
|---|---|---|---|
| G(1606)T | Caucasians | Chinese | Africans |
| n % | 98 | 91 | 94 |
| GG | 32 (32.3%) | 54 (59.3%) | 75 (79.8%) |
| GT | 52 (52.5%) | 35 (38.5%) | 18 (19.2%) |
| TT | 15 (15.2%) | 2 (2.2%) | 1 (1%) |
| % G | 59.2 | 78.6 | 89.4 |

This genotype distribution differs in a highly significant manner in the Chi² test with Chi of 51.0 and p<0.0001. The GG genotype occurs most frequently in black Africans.

TABLE 3

| Intron 1 | White | | Black |
|---|---|---|---|
| C(10564)T | Caucasians | Chinese | Africans |
| n % | 98 | 84 | 86 |
| CC | 46 (46.5%) | 28 (33.3%) | 39 (45.3%) |
| CT | 43 (43.3%) | 42 (50.0%) | 36 (41.9%) |
| TT | 10 (10.1%) | 14 (16.7%) | 11 (12.8%) |
| % C | 68.2 | 58.3 | 66.3 |

This genotype distribution does not bring to light any significant difference between the ethnic groups in the Chi² test with Chi of 4.4 and p=0.352. The CC genotype occurs most frequently in black Africans while in Chinese the heterozygous CT carrier is most prevalent. From this distribution, the conclusion can be drawn that, from the historic development point of view (based on Caucasians), GG(−659) and GG(1606) represent the “original state”. Such differences in the genotype distribution in the case of different ethnic groups usually point towards associated phenotypes having been significant for evolution and provided a specific advantage to the carrier. It is known to the expert that ethnically different genotype distributions provide an indication that, even today, certain genotypes and haplotypes are associated with certain diseases or physiological and pathophysiological types of reaction or responses to therapy, e.g. with drugs.

The object of the invention consists also of these polymorphisms being used for detecting and validating further relevant genomic gene modifications in GNA11 or neighbouring genes which, for example, are in coupling imbalance with genotypes in gene GNA11. These may also be genes which are equally positioned on chromosome 19, but at a large distance from the gene GNA11. The following procedure is used for this purpose:

### 1. For certain phenotypes. (cellular properties, disease conditions, disease developments, drug responses etc.) an association is initially produced with the polymorphisms G(−659)C, G(1606)T or C(10564)T.

### 2. For newly detected gene modifications in GNA11 or neighbouring genes, an investigation is carried out whether associations already existing are strengthened or weakened by using the above-described genotypes or haplotypes.

### Haplotype Analyses of the Promoter Polymorphism G(−659)C with the Intron I Polymorphisms G(1606)t and C(10564)t

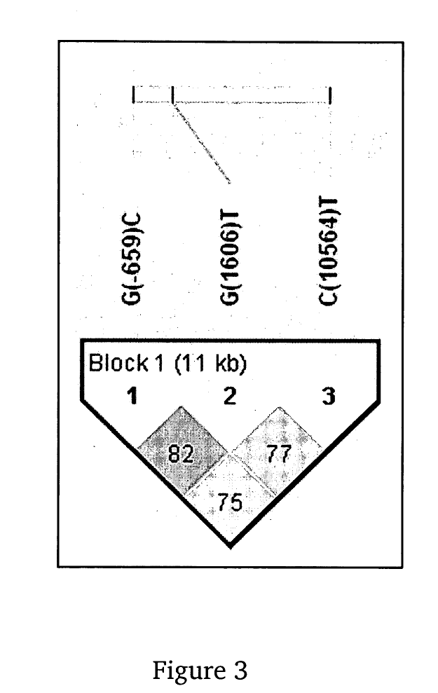

In order to be able to express an opinion as to whether the three above-mentioned polymorphisms are transmitted together and in which coupling imbalance they are vis-à-vis each other, a haplotype analysis was carried out using the “Haploview” computer program. This made it clear that the promoter polymorphism G(−659)C is coupled 82% to G(1606)T and 75% to C(10564)T. The two intron I polymorphisms G(1606)T and C(10564)T couple 77% to each other (FIG. 3). This means that it is not always sufficient to examine only one of the three above-mentioned polymorphisms in order to be able to draw conclusions regarding the others. For this, an almost 100% coupling would be necessary.

### Functional Significance of the G(−659)C Polymorphism

An investigation was carried out as to which functional modifications need to be allocated to the genotypes of the polymorphisms in gene GNA11 described here. A tissue-specific expression or an overexpression of the Gα11 protein as a function of the genotypes of the G(−659)C polymorphism or the other polymorphisms mentioned, for example, is conceivable in this case. For this purpose, an investigation was first carried out by means of a computer program whether the nucleotide exchange is capable of influencing the binding of transcription factors.

Transcription factors bind to specific consensus sequences and can increase or reduce the promoter activity such that an increased or reduced transcription of the gene results and consequently the expression level of the encoded protein is increased or reduced. The G(−659)C polymorphism is present in a consensus sequence of the binding site for the transcription factors AP2-alpha, Myf, SPI-1 and MZF 1-4 whose binding capacity can be negatively affected by the polymorphism. This impairment relates mainly to the binding capacity of the transcription factor AP2-alpha onto the genotype G(−659). The occurrence of this genotype leads to a weaker bond or an elimination thereof to the consensus sequence: TCCCCAGGC. AP2-alpha can have an inhibitory effect on the expression of genes (Jiang et al., The repressive function of AP2 transcription factor on the hepatocyte growth factor gene promoter. Biochem Biophys Res Commun. 2000 Jun. 16; 272(3):882-6; Ren and Liao, Transcription factor AP-2 functions as a repressor that contributes to the liver-specific expression of serum amyloid A1 gene. J Biol Chem. 2001 May 25; 276(21):17770-8.)

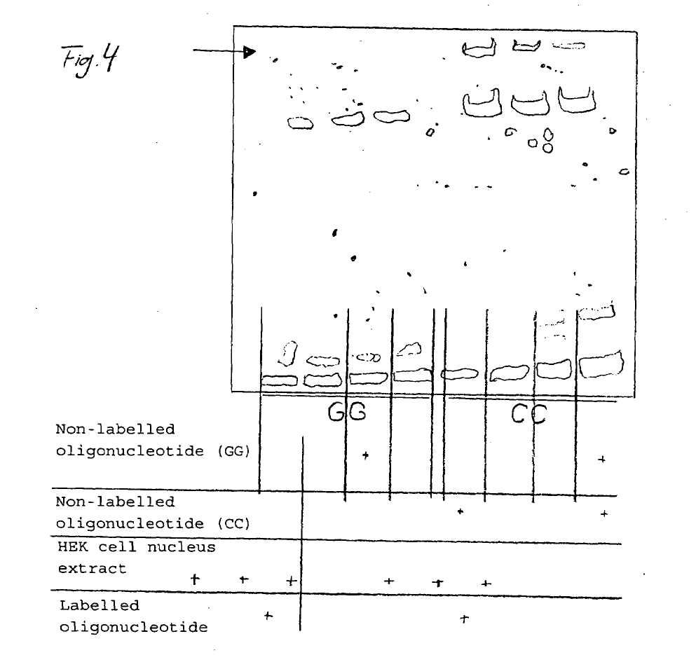

For an experimental investigation of this effect, a so-called EMSA (electrophoretic mobility shift assay) is carried out. In the case of this experiment, short nucleic acid sections containing the polymorphism are incubated with cell nucleus extracts. Transcription factor proteins which are present in the extracts then bind with different intensities to the nucleic acid sections. The binding to DNA is finally made visible on the X-ray film. An intensive band results in this case from strong bond. FIG. 4 shows the result of this experiment with specific constructs containing either the G or the C genotype. The stronger intensity of the C construct band proves a stronger binding of a transcription factor to this region is present.

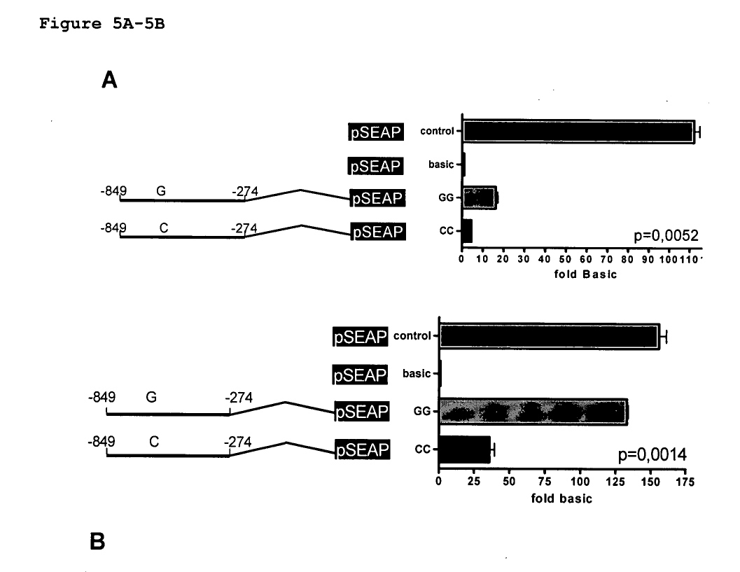

For the functional detection of a modified promoter activity as a function of certain genotypes, different fragments of the promoter were cloned with the G or the C genotype in vector PSEAP in order to quantify, following the expression of the vector in HEK cells, the promoter activity by means of a so-called “reporter assay” (FIG. 5). For this purpose, the constructs are cloned in front of a gene which codes for secreted alkaline phosphatase (SEAP). If the construct has a promoter activity, the SEAP gene transcription is increased and the increased secretion of alkaline phosphatase into the cell culture medium is measurable. As shown in FIG. 5, the construct −849/−274 exhibits a high promoter activity. Since the polymorphism is present in this region and, moreover, a transcription factor binding site is influenced by the nucleic acid exchange, an investigation was carried out to determine to what extent the promoter activity of the construct behaves as a function of the genotype.

For this purpose, the construct −849/−274 was transfected in each case with the allele G(−659) and C(−659) in HEK and Hela cells. In the case of constructs with the G allele, the promoter activity is increased 4-5 fold, significantly (p<0.05) in comparison with the C-allele. The G polymorphism in the promoter of the GNA11 gene also leads to the promoter activity being increased and consequently the Gα11 protein being expressed in an increased manner.

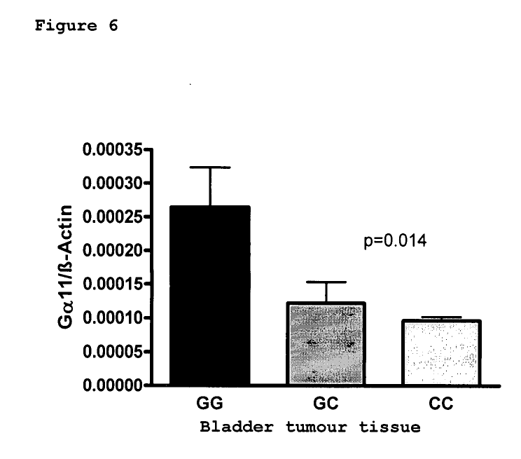

In order to check whether this regulation also takes place in vivo the expression of Gα11 was examined at mRNA-level using real time PCR in bladder tumours. For this purpose, mRNA was obtained from human surgical tissue during surgery and transcribed by reverse transcriptase into cDNA. The expert is familiar with this process. Subsequently, the expression level was determined by means of real time PCR (Taqman method) and compared with the expression level of the housekeeping gene βM-actin. The results are shown in FIG. 6. The GG genotype leads to an increase in the Gα11 transcription of at least 20% compared with the C allele.

The process according to the invention is used for the prognosis, diagnosis and therapy of the following diseases. These diseases are accompanied by a modified expression level of the Gα11 protein.

1. Cardiovascular diseases. These include in particular high blood pressure, stroke, coronary heart disease and myocardial infarction, heart failure, irregular pulse, preclampsia or toxaemia of pregnancy,
2. Enocrinological and metabolic disorders. These include in particular adipositas, metabolic syndrome, type-2 diabetes mellitus, gout, osteoporosis, thyroid diseases such as hyperthyreosis and hypothyreosis and M. Basedow, hyperparathyroidism and hypoparathyroidism, Cushing's Disease, hyperaldosteronism and hypoaldosteronism,

### 3. Psychiatric illnesses such as depression, schizophrenia, alcoholism and anxiety disorders, phobias, neuroses,

### 4. Neurological diseases such as Parkinson's disease, multiple sclerosis, epilepsies,

### 5. Dermatological diseases such as psoriasis, neurodermatitis,

### 6. Tumour diseases.

As an example, the use of gene modifications in gene GNA11 will be discussed below for predicting the risk of cardiovascular diseases.

In the case of transgenic animals which overexpress Gα/11 in the heart, an increased tendency towards cardiac hypertrophy, heart failure and apoptosis is observed. It has therefore been used as a starting point that an increased expression of Gα11 in man, such as it occurs in the case of the GG genotype of the G(−659)C polymorphism, will lead to an increased cardiovascular risk.

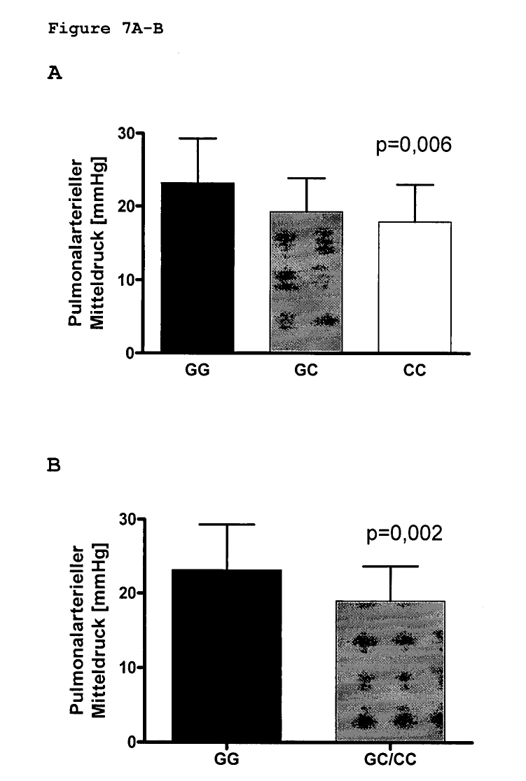

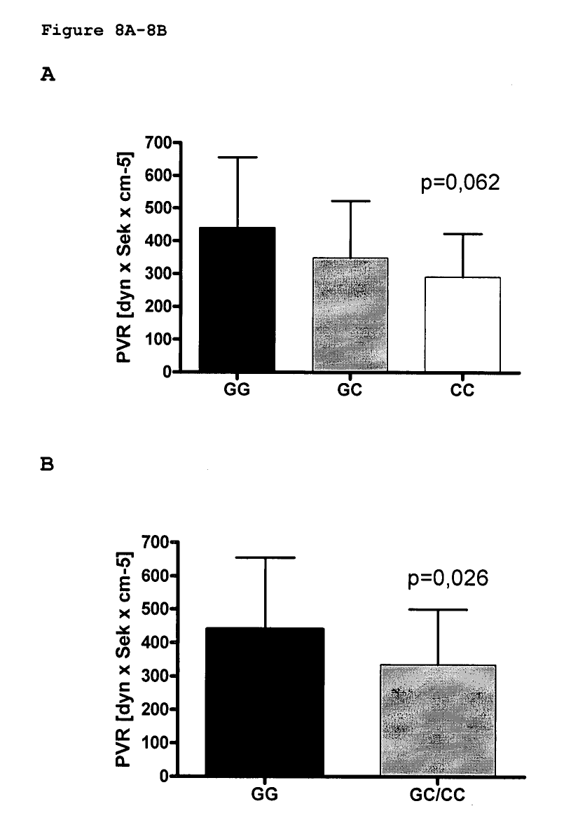

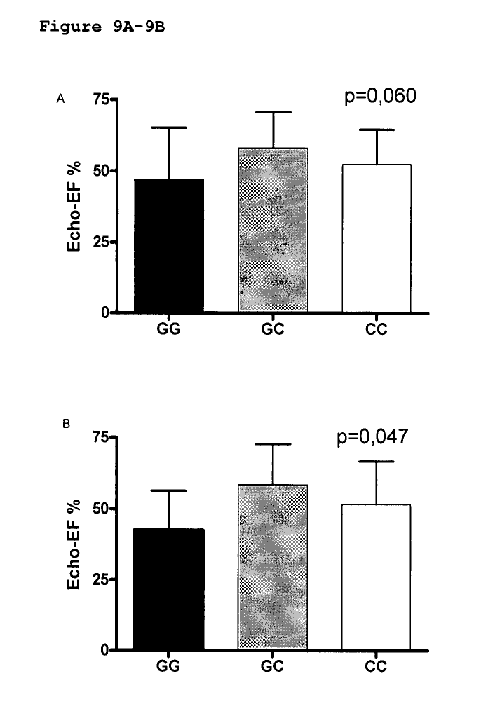

As illustrated in FIG. 7, the pulmonary arterial mean pressure in patients with coronary heart disease who are homozygous for the GG genotype, is increased significantly (p=0.006) in comparison with the CC genotype. For the pulmonary vascular resistance (FIG. 8), it has also been possible to determine an almost significant difference (p=0.062) between the GG and the CC genotype. On considering the GG/CC allele carrier in combination, the values became significant with p=0.026. During echocardiography, clear genotype-dependent differences can be shown in the ejection fraction in the case of patients who have already suffered a heart attack (p=0.047)(FIGS. 9 A and B). Thus there is a better prognosis for C allele carriers following a heart attack with an ejection fraction of approximately 52% than for G-allele carriers with a restricted ejection fraction of hardly 43%. Overall, a clearly increased cardiovascular risk can thus be allocated to the (−659)G allele carriers.

The process according to the invention can be used for pharmacogenetic purposes, i.e. for the diagnosis of the effectiveness of drugs, the potency and efficiency of drugs and the occurrence of undesired effects.

The effectiveness of pharmaceuticals and/or the occurrence of undesired side effects is defined by a number of parameters, apart from the specific substance properties of the chemically defined products. Two important parameters, namely the achievable plasma concentration and the plasma half-life, determined to a large extent the effectiveness or ineffectiveness of drugs or the occurrence of undesired effects. The plasma half-life is determined, among other things, by determining the speed with which certain drugs are metabolised in the liver or other body organs into effective or ineffective metabolites and the speed with which they are excreted from the body, it being possible for the excretion to take place via the kidneys, via the respiratory air, via sweat, via seminal fluid, via faeces or via other body secretions. In addition, the effectiveness on oral administration is limited by the so-called “first pass effect” since, following the resorption of drugs via the bowel, a certain proportion is metabolised in the liver into ineffective metabolites.

Mutations or polymorphisms in genes of metabolising enzymes can change the activity thereof in such a way that their amino acid composition is modified as a result of which the affinity to the metabolising substrate is increased or reduced and consequently the metabolism can be accelerated or slowed down. In a similar manner, mutations or polymorphisms in transport proteins can modify the amino acid composition in such a way that the transport and consequently the excretion from the body is accelerated or slowed down.

To choose the substance which is suitable in an optimal manner for a patient, the optimum dosage, the optimum form of administration and to avoid undesired, in some cases harmful or fatal side effects, knowing genetic polymorphisms or mutations leading to modification of the gene products is of outstanding importance.

### The Effect of Hormones in the Human Body and the Significance of Polymorphisms in Hormone Receptors

Numerous hormones and peptide hormones of the human body, but also receptor antagonists, exert their effect on so-called receptors of the body cells. These consist of proteins of different composition. Following the activation of these receptors, these signals must be directed into the cell interior mediated via the activation of the heterotrimeric G-proteins. Such G-proteins are composed of different α, β and γ-subunits. Depending on the activatability by defined hormones, these receptors can be subdivided into specific groups. The expert is aware that mutations or polymorphisms in certain receptors are capable of determining the effectiveness of certain agonists or antagonists on these receptors. Thus, a frequent Gly16Arg polymorphism in the gene coding for the β2 adrenoreceptor influences the intensity of the responsiveness to the β2 sympathomimetic salbutamol (Martinez F D, et al. Association between genetic polymorphisms of the beta2-adrenoceptor and response to albuterol in children with and without a history of wheezing. J Clin Invest. 1997 Dec. 15; 100(12):3184-8). Polymorphisms in the D2 receptor gene determine the frequency of the occurrence of dyskinesias during the treatment of Morbus Parkinson (Parkinson's disease) with Levadopa (Oliveri R L, et al.; Dopamine D2 receptor gene polymorphism and the risk of levodopa-induced dyskinesias in PD. Neurology. 1999 Oct. 22; 53(7):1425-30): Polymorphisms in the popiate receptor gene determine the analgetic effectiveness of opiates (Uhl G R, et al. The mu opiate receptor as a candidate gene for pain: polymorphisms, variations in expression, nociception, and opiate responses. Proc Natl Acad Sci USA. 1999 Jul. 6; 96(14):7752-5).

The gene modifications in specific receptors mentioned can only be used for the diagnosis of the effect of drugs to the extent that these drugs are specific agonists or antagonists on the receptors considered. However, an individual diagnosis of the general responsiveness vis-à-vis all drugs and the individual prediction of the risk of undesired effects during the therapy with drugs is desirable.

### The Diagnosis of the Activatability of the Gα11 Protein Allows a General Diagnosis to be made of the Effectiveness of Drugs, Their Optimum Dosage and the Occurrence of Side Effects.

Drugs should be understood to mean in general sub-stances which can be externally supplied to the body in order to produce specific states. Such substances can be hormones, low or high molecular substances, peptides or proteins, antibodies.

Most drugs used for treating diseases, bodily malfunctioning or disturbances of the sense of well-being are hormones, agonists on hormone receptors, antagonists on hormone receptors or other substances which influence the expression of receptors or the concentration of hormones directly or indirectly. A number of drugs exert this influence by physiological counter-regulations taking place during the therapy with such substances, which counter-regulations increase the concentration of hormones which activate the G-protein-coupled receptors. As a generally known example, the therapy with urinative substances (diuretics), in particular loop diuretics and thiazide diuretics, deserves to be mentioned. The loss of sodium chloride and the decrease in blood pressure occurring in the course of the therapy lead to the activation of the renin/angiotensin/aldosterone system. The hormone angiotensin II formed in increased amounts stimulates an increased resorption of sodium in the kidney, stimulates the salt absorption, increases the blood pressure by a direct vasoconstrictory effect onto smooth vascular muscle cells and induces proliferation processes. It is generally known that these mechanisms caused by angiotensin II occur after coupling of the hormone to receptors which mediate their effect via an activation of heterotrimeric G-proteins. The efficiency of these effects is predictable if the strength of the activatability of G-proteins can be diagnosed. Other drugs exert their effect by inhibiting the re-uptake of transmitters liberated from neurons such as noradrenalin, adrenaline, serotonin or dopamine. The drug sibutramine can be mentioned here as an example which inhibits the re-uptake of serotonin and noradrenaline in the central nervous system thus reducing the sensation of hunger and thermogenesis. Correspondingly, sibutramine can be used for the therapy of adiposity. Since noradrenaline and serotonin activate G-protein coupled receptors, the diagnosis of the activatability of G-proteins is particularly suitable for predicting the effectiveness of sibutramine and the occurrence of typical, sibutramine-associated side effects (e.g. increase in the heart rate and blood pressure).

According to the invention, a process is provided which is suitable for the general diagnosis of the activatability of G-proteins. For this purpose, one or several polymorphisms in the gene GNA11 which encodes the human Gα11 subunit of heterotrimeric G-proteins are examined. In the case of an overexpression, an increased activatability of heterotrimeric G-proteins and an increased activatability of all cells of the human body predictably occurs. In this way, a determination of the presence of polymorphisms in GNA11 permits the diagnosis of the effectiveness and undesired effects of pharmaceuticals, in particular agonists and antagonists of all receptors whose effect is mediated via heterotrimeric G-proteins. In addition, such polymorphisms in GNA11 can be used to diagnose the effect of drugs which increase or reduce the concentrations of endogenous hormones either indirectly or as a result of counter-regulation mechanisms, the receptors of which hormones activate heterotrimeric G-proteins. Thus the invention permits a diagnosis of the effects and undesired effects of all drugs and is not limited to drugs which influence specific receptors in an agonistic or antagonistic manner. In addition, the diagnosis of the allele or haplotype status in GNA11 can be used to determine the individually optimal and tolerable dosage of pharmaceuticals.

For the diagnosis of an increased or reduced activatability of G-proteins, the detection of the G(−659)C polymorphism is used in particular. In addition, all further gene modifications in GNA11 can be used for diagnosis which are in a coupling imbalance to this polymorphism or additionally promote or inhibit the expression.

The above-mentioned process is suitable in particular for the diagnosis of the effect of agonists or antagonists on receptors whose effect is known to be mediated by G-proteins. In this connection, the following examples are mentioned, the list of the examples not being complete:

### 1) Adrenergic receptors, in particular α and β adrenoceptors and their isoforms and subgroups, i.e. α1- and α2-adrenoceptors as well as β1, β2, β3 and β4 adrenoceptors;

2) Muscarine receptors and their isoforms, e.g. m1-, m2-, m3-, m4 and m5-muscarine receptors and their subtypes. Typical antagonists on muscarine receptors are e.g. atropine, scopolamine, ipratroprium, pirenzepine and N-butyl scopolamine. Typical agonists are carbachol, bethanechol, pilocarpine;
3) Dopamine receptors, e.g. D1, D2, D3, D4 and D5 receptors and their isoforms and splicing variants;
4) Serotonin receptors, e.g. 5-HT1, 5-HT2, 5-HT3, 5-HT4, 5HT-5, 5HT-6 and 5-HT7-receptor and their subtypes, isoforms and splicing variants. Typical agonists are sumatriptan and cisaprid, antagonists are e.g. ondansetrone, methysergide, buspirone and urapidil;

### 5) Endotheline receptors and their subtypes, isoforms and splicing variants;

### 6) Bradykinin receptors such as B1 and B2-receptors and their subtypes, isoforms and splicing variants;

7) Angiotensin receptors e.g. AT II Type 1 and Type 2 receptor, typical antagonists on the AT II receptor are losartane and other sartanes;

### 8) Receptors for endorphines and opiates such as the μ-opiate receptor;

### 9) Chemokine receptors CCR1-12 and CXCR1-8 for e.g. interleukin-1/2/3/4/5/6/7/8/9/10/11/12, RANTES, MIP-1α, MIP-1β, stromal cell-derived factor, MCP1-5, TARC, lymphotactin, fractalkine, eotaxin 1-2, NAP-2, LIX;

### 10) Adenosine receptors and their subtypes, subforms and splicing variants;

### 11) Receptors for thrombin (protease-activated receptors);

### 12) Receptors for lyso-phosphatidic acid, phosphatidic acid, receptors for sphingosine phosphate and their derivatives;

### 13) Receptors for prostaglandins and thromboxanes such as for PGE1, PGE2, PGF, PGD2, PGI2, PGF2α, thromboxane A2;

### 14) Receptors for neuropeptides such as NPY1-5;

### 15) Histamine receptors such as H1-H3 receptors;

### 16) Receptors for the platelet activated factor (PAF receptor);

### 17) Receptors for leukotrienes;

### 18) Receptors for insulin, glucagon, insulin-like growth factor (IGF-1 and IGF-2), epidermal growth factor (EGF) and platelet-derived growth factor (PDGF);

### 19) Receptors for growth hormone (GH), somatostatin (SSTR1-5), thyreotropic hormone (TSH), oxytocin, prolactin, gonadotropins;

### 20) Receptors for zytokines such as interferons;

### 21) Receptors for purines;

22) Orphan receptors whose effect is mediated by G-proteins. Receptors for leptin; and

### 23) CpG—oligo nucleotides.

In addition, the effects of drugs can be predicted which influence the re-uptake, degradation or renewed synthesis of neurotransmitters or in the case of which modifications occur during therapy in the expression or responsiveness of the above-mentioned receptors (e.g. sibutramine, fluoxetine). Moreover, the effects of all drugs are diagnosed which modify, directly or indirectly, the concentration of agonists activating the above-mentioned receptors also as a result of a physiological counter-reaction. Moreover, the influence of radiation therapy on cancer patients can also be predicted.

The effects and undesired effects of the following drugs, in particular, can be diagnosed from the following indication ranges: Antihypertensives, e.g. β-blockers (propanolol, bisprolol, etc.), diuretics (hydrochlorothiazide and other thiazide diuretics; furosemide, piretanide and other loop diuretics, chlorothalidone), α1-adrenoreceptor blockers (e.g. doxazosin, prazosin), angiotensin receptor blockers (e.g. losartan), ACE-inhibitors (enalapril, captopril, ramipril etc.), Ca²⁺ channel blockers (e.g. nifedipine, verapamil, amlodipine, felodipine), clonidine, reserpine, renin inhibitors. Drugs for treating heart failure, e.g. β-blockers (e.g. propanolol, metoprolol), ACE inhibitors (e.g. captopril, enalapril, ramipril, etc.), angiotensin receptor blockers (e.g. losartan), digitalis glykosides, katechin amines, diuretics. Drugs for treating low blood pressure or heart failure, e.g. α and β-sympathomimetics (effortil, adrenaline, noradrenaline, dobutamine, β-adreno receptor blockers, ACE inhibitors, angiotensin II receptor blockers.) Drugs for treating migraine, e.g. sumatriptan, rizatriptan, zolmitriptan and other agonists on serotonin receptors, β-blockers (propanolol, timolol), ergotamine and dihydroergotamine. Analgesics of the morphine type (morphine, codeine etc.). Drugs for treating coronary heart disease such as adenosine, β-blockers (e.g. propanolol, acebutolol), nitrates and Ca²⁺ channel blockers. Drugs for treating psychiatric illnesses (schizophrenia, manic depressive illnesses, psychoses, depressions) and of addiction diseases such as alcoholism (e.g. fluoxetine, paoxetine, imipramine, desipramine, doxepine, mianserine, trazodon, lofepramine), anxiety syndromes (diazepam etc.) which influence e.g. the dopaminergic, serotonergic or adrenergic system. But also drugs which mediate their effect via receptors for GABA, glycine or glutamate or their derivatives. Drugs for treating Alzheimer's disease (e.g. tacrine) and for treating Parkinson's disease (e.g. bromocriptin, L-DOPA, carbidopa, biperiden, seleginil etc.) which influence transmitter concentrations on e.g. muscarinergic or dopaminergic substances. Drugs for treating bronchial asthma which have either a direct broncho-dilating or anti-inflammatory effect, e.g. salbutamol, terbutalin, albuterol, theophyllin, montelukast, zafirlukast, cromoglicinic acid, ipratropium bromide. Such drugs include also antibodies directed against specific proteins and receptors. Drugs for treating motility disorders of the stomach or bowels and drugs for treating irritable bowel syndrome, e.g. N-butyl scopolamine, pirenzepin, metoclopramide). Drugs for treating adiposity which activate either directly lipolytically effective receptors, e.g. β3-adrenergic agonists or have a central effect, e.g. sibutramine or similar substances which modify the feeling of satiety or which influence thermogenesis These include drugs which influence gastric emptying. Drugs for treating chronic inflammation processes or disorders of the immune system, e.g. zytokines (interferons) in the therapy of virus hepatitides or interleukin-2 in the case of HIV infection. Such diseases include Crohn's disease, ulcerative colitis, asthma, psoriasis, neurodermatitis, hay fever. They also include antibodies to zytokines or to zytokine receptors e.g. to TNFα. Drugs for treating toxaemia of pregnancy/eclampsia and the HELLP syndrome. Drugs for treating fertility problems or for eliminating menstrual cycle irregularities in women or for birth control. Drugs for treating irregular pulse. Antidiabetics (acarbose, insulin, troglitazone, metformin etc.). Hypnotics, anti-emetics and anti-epileptics. Drugs for the treatment of sexual problems, e.g. erectile dysfunction, female sexual dysfunction, lack of libido, orgasmic disorders (phosphodiesterase inhibitors such as sildenafil, prostaglandin E1, agonists to dopamine receptors, e.g. apomorphine, yohimbin, phentolamine). Drugs for the therapy of cancer diseases and chemotherapeutics, e.g. 5-fluorouracil, antibodies to proteins and receptors (e.g. to HER-2), substances blocking tyrosine kinases etc. Drugs for treating allergic and tumour diseases in which the effect is achieved via the administration of CpG nucleotides. Drugs for treating adiposity, metabolic syndrome or diabetes, e.g. sibutramine, orlistat, leptin, topiramat, glinidins, glitazones, biguanides etc. Drugs for treating HIV-infection, also antibodies and receptor blockers, prediction of the occurrence of lipodystrophy during therapy with proteinase inhibitors.

Obviously, it is not possible to provide proof within the framework of the invention described here that all drug effects are determined by the GNA11 gene status. Naturally, it is also not possible to investigate the genotype-dependent effects of drugs which will be developed and used in the future. On the other hand, examples of drugs with different mechanisms of action are to be shown here such that these findings can be generalised.

In the case of the drug (sibutramine), the effect is caused by the re-uptake of serotonin and noradrenaline in the central nervous system being blocked. As a result, the concentration of these transmitters in the synaptic cleft and in the extracellular region is increased. These transmitters subsequently simulate G-protein-coupled receptors. The substance sibutramine itself does not stimulate such receptors.

A major object of the invention is the provision of diagnostically relevant gene modifications in the gene GNA11 as a prognosis factor for predicting the course of all tumour diseases in man.

In general terms, all cells of the human body may degenerate in a malignant manner and lead to cancer disease. The details provided here and in the following describe general mechanisms of tumour progression, metastasis and the therapeutic response. To this extent, the mechanisms and claims described here apply to all tumours in man, e.g. the following tumours: Tumours of the urogenital tract: here, the bladder carcinoma, the kidney cell carcinoma, the prostate carcinoma and the seminoma deserve to be mentioned. Tumours of the female reproductive organs: the mammary carcinoma, the corpus carcinoma, the ovarian carcinoma, the cervic carcinoma. Tumours of the gastrointestinal tract: the oral cavity carcinoma, the oesophagus carcinoma, the stomach carcinoma, the liver carcinoma, the bile duct carcinoma, the pancreas carcinoma, the colon carcinoma, the rectum carcinoma. Tumours of the respiratory tract: the larynx carcinoma, the bronchial carcinoma. Tumours of the skin: malignant melanoma, the basalioma, the T-cell lymphoma. Tumour diseases of the haemopoietic system: Hodgkin- and non-Hodgkin lymphoma, acute and chronic leukaemia etc. Tumour diseases of the brain and/or the nerve tissue: glioblastoma, neuroblastoma, medulloblastoma, meningeal sarcoma, astrozytoma. Soft tissue tumours, e.g. sarcomas and head-neck tumours.

The following examples serve for further illustration of the invention.

### EXAMPLE 1

### Effectiveness of Sibutramine

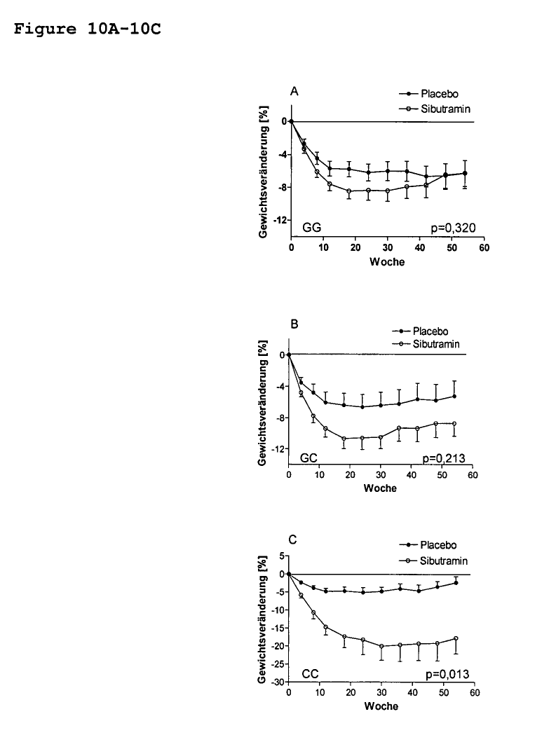

In this case, adipose patients were treated for one year with 15 mg of sibutramine/day within a placebo controlled, double blind study. Sibutramine is a centrally effective re-uptake inhibitor of noradrenaline and serotonin, which both develop their effect via G-protein-coupled receptors. Sibutramine increases the feeling of satiety and facilitates weight loss within the framework of structured weight reduction measures (increase in bodily activity, reduction of calorie consumption). The clinical end target of the study was the weight loss after one year. The genotype-dependent comparison of placebo vis-à-vis sibutramine is illustrated in FIG. 10. In the sibutramine group, patients with the GNA11-659 CC genotype were able to lose weight clearly better (17.8%) than those with the GG or GC genotype (6.2%/8.7%). The administration of sibutramine leads to a marked (p=0.013) increase in weight loss only in the case of carriers of the CC genotype, whereas this advantageous pharmaceutical effect was not observed in patients with the GG genotype. Consequently, a therapy with sibutramine is indicated pre-dominantly for patients with CC genotype whereas patients with GG genotype are able to lose weight just as easily with a placebo (6.3%) as with sibutramine (6.2%).

In the placebo group, patients with the GNA11 (−659)GG or GC genotype were able to lose weight markedly better (6.3% and 5.2%) than those with the CC genotype (2.4%). The administration of sibutramine leads to a marked (p=0.013) increase in weight loss only in the case of carriers of the CC genotype, whereas this advantageous pharmaceutical effect was not observed in patients the GG genotype. Consequently, a therapy with sibutramine is indicated predominantly for patients with the CC genotype, whereas patients with the GG- or GC genotype are capable of losing weight also with a placebo. Consequently, it is shown simultaneously that the prognosis of success of non-pharmacological measures for weight reduction is possible by using gene modifications in the gene GNA11. This includes a structured weight loss programme (e.g. Optifast, Weight Watchers, other training programmes), the consumption of sating swelling substances (CM3, BMI23) or of calorie-reduced foods.

### EXAMPLE 2

### Prognosis of Cardiovascular Side Effects During Therapy with Sibutramine

As a result of the mechanism of action (central inhibition of the re-uptake of noradrenaline and serotonin) typical side effects such as a dry mouth, sleep disturbances, obstipations occur in the case of the consumption of sibutramine. However, the cardiovascular side effect s such as an increase in the heart rate and blood pressure which can lead to tachycardia and myocardial infarction are more dangerous. So far, no prognosis in this respect is possible as to the persons in whom this will occur.

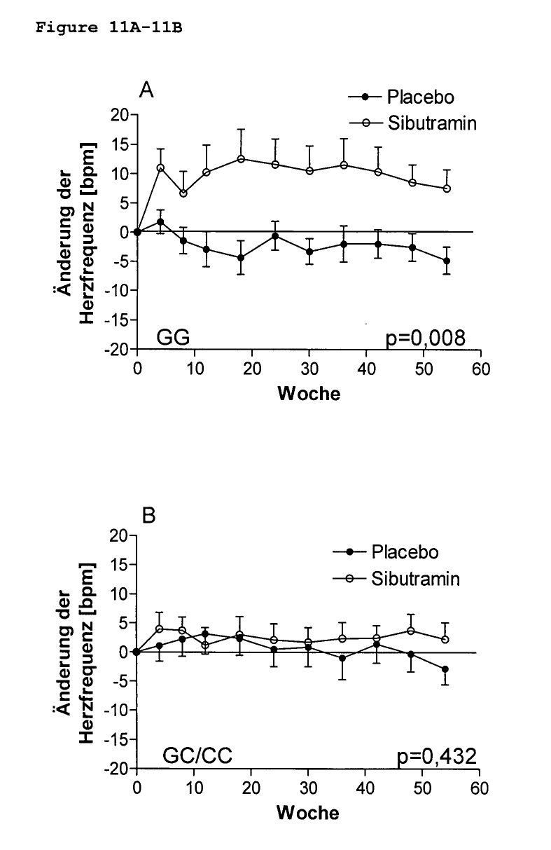

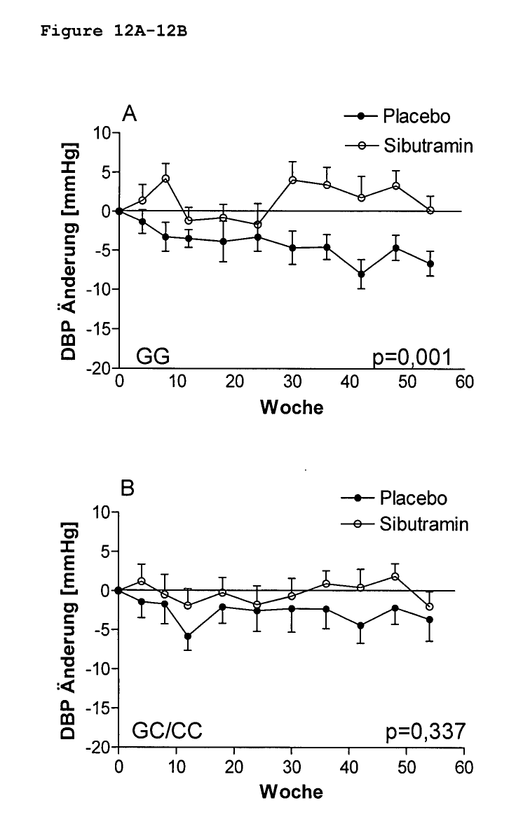

The connection between gene modifications in the gene GNA11 and the change in the heart rate when taking sibutramine was investigated vis-à-vis a placebo (FIG. 11). A significantly increased heart rate (p=0.008) by >10 (GG) up to 17 beats per minute can be seen in the case of G(−659)C and GG genotypes (FIG. 11A). This effect does not occur in the case of CC and GC genotypes (FIG. 11B). In addition, a significant increase in the diastolic blood pressure (p=0.001) during therapy with sibutramine is observed in the case of (−659) GG genotypes (FIG. 12A) which does not occur in the case of CC and GC genotypes (FIG. 12B).

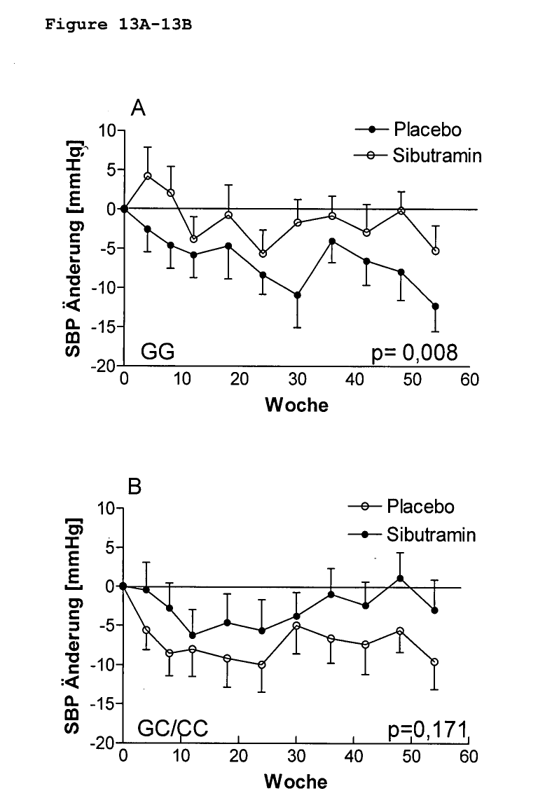

In the case of a therapy with sibutramine, a lacking decrease in the systolic blood pressure in the case (−659) GG genotypes (FIG. 13A) is observed, the difference in the blood pressure change of p=0.008 being significantly higher than in the case of GC/CC genotypes (FIG. 13B).

These observations are therapeutically significant because it has been shown that precisely patients with GG genotypes lose sufficient weight by a calorie-reduced diet and a change in lifestyle alone, these consequently not benefiting from a therapy with sibutramine.

The use of gene modifications is consequently suitable for identifying patients in whom cardiovascular side effects occur during therapy with medicines. These effects can be caused directly in that such medicines stimulate receptors which cause a vasoconstriction or a heart rate increase mediated by Gα11. These include sibutramine, triptanes and noradrenaline/serotonin re-uptake inhibitors, for example. In addition, these include medicines which inhibit the breakdown of katechin amines (MAO-inhibitors) and tricyclic antidepressants. Moreover, they include medicines which effect a decrease in blood pressure and induce, by reflex, an activation of the sympathetic nervous system (e.g. sildenafil and other inhibitors of phosphodiesterases, nitrates).

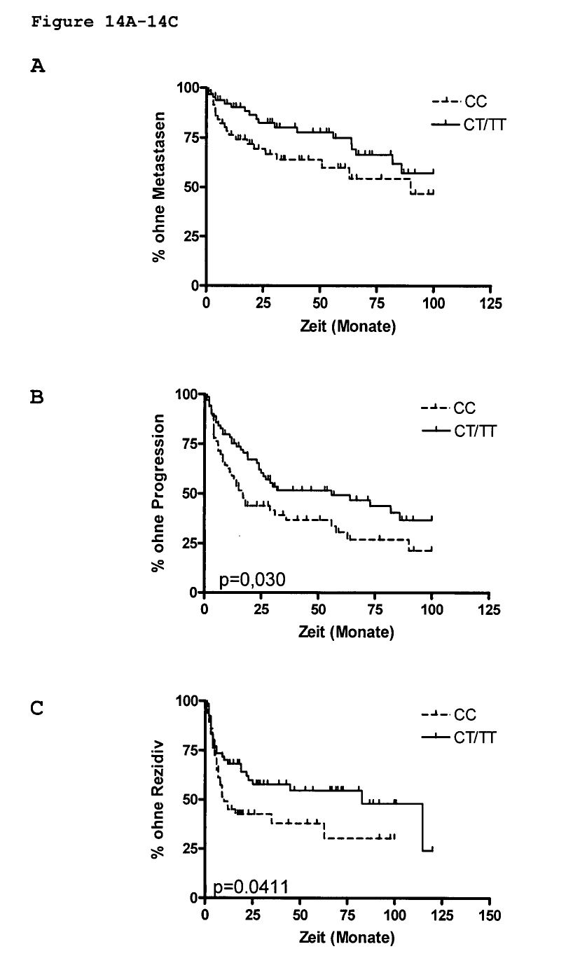

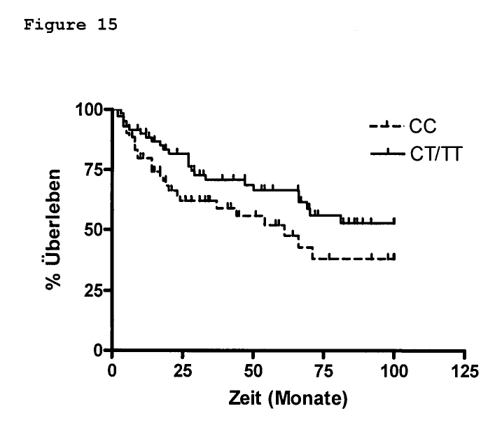

A further piece of evidence for the general application of gene modifications in the gene GNA11 for the prognosis of the drug effects is obtained also from the genotype-dependent disease developments in the case of the bladder carcinoma observed for the C(10564)T polymorphism (FIGS. 14 and 15). These patients were all treated with different drugs. The different causes of the disease made visible by using gene modifications in the gene GNA11 provide proof of a varying response to this form of therapy.

### Basic Properties of Malignant Tumours

In the case of malignant tumours, also known as cancer, characteristic modifications of basic functions which promote the growth of such cells in an unfavourable manner occur. Cancer cells are characterised by a loss of contact inhibition and an uncontrolled cell growth. Such modifications are triggered by numerous noxae, so-called carcinogens, which damage the genotype. Such noxae include many chemicals, tobacco smoke but also UV-light. In addition, genetic factors play a major part in the formation of cancer. A characteristic feature of cancer cells, apart from their uninhibited growth, is also the tendency to deposit metastases in other organs. The spread of metastases takes place regularly via the blood flow or lymph vessels. Cancer diseases are incurable in the majority of cases and lead to death. Therapeutically, an attempt is made to remove the initial tumour and metastases surgically. In addition, tumours can be eradiated. By means of so-called cytostatics, antibodies against certain proteins or surface markers or immuno-modulating sub-stances (cytokines, interferons) an attempt is made to kill the rapidly dividing cancer cells or to subject them to programmed cell death (apoptosis). The therapeutic measures available at present lead merely to a prolonged survival in most cases, but not to definitive healing.

### Use of Gene Modifications in the Gene GNA11 for the Prognosis of the Development of Tumour Diseases

Naturally, it is not possible to describe all tumour diseases here. The principle is consequently illustrated by way of a selected example which demonstrates the general usability:

### EXAMPLE 3

### Bladder Carcinoma

Bladder carcinoma is a malignant tumour of the mucous membrane of the bladder. A bladder carcinoma occurs most frequently between the 60^{th} and 70^{th} year of life. Men are affected three times as frequently as women. In men, bladder carcinoma is the third most frequent form of cancer after lung and prostate cancer. Bladder carcinoma can be caused by external influences. The risk factors include smoking, ongoing stress on the organism by chemicals such as dyes, misuse of pain killers. In the case of many patients, examinations show that a surface tumour is involved. This can be removed surgically by means of the cystoscope. More than 70% of the patients treated for a surface bladder carcinoma will have a recurrence of the tumour subsequently. In more than half of the cases, recurrent tumours occur with a non-muscle-invasive disease. These can be treated therapeutically or kept in check by transurethral resection. It is consequently important to recognise these lesions early on and to provide regular close after-care for the patient. In this respect, cystoscopy with urine cytology is the most important aspect. Excretion urography at regular intervals is used to check possible tumour manifestations in the renal pelvis and urethra. So far, hardly any valid markers exist which are predictive of the further development of the disease. Consequently, at present, conventional factors such as the depth of penetration, the degree of differentiation, metastases, lymph node attack etc. are used for prognosis. Genetic markers for tumour progression, the tendency to relapse, the survival probability and therapy responses would considerably improve the care of patients with bladder carcinoma. A further component of the invention consists of the fact that the use of gene modifications in GNA11 is suitable for predicting the further course of the disease. FIG. 14 (top) shows the point in time up to the occurrence of metastases as a function of the GNA11 C(10564)T polymorphism. The metastasis risk is increased in this case by approximately ⅓ in patients with C-allele. A similar relationship is detected if the time up to tumour progression is investigated (FIG. 14 (middle)). This should be understood to mean the occurrence of metastases or the reoccurrence of the tumour with increased staging or grading. The course of the curve is significantly different for the genotypes (p=0.030, Logrank test) the CT/TT geno-type carriers being allocated the more advantageous course. Finally, the connection between the GNA11 C(10564)T TT polymorphism and the formation of a relapse is illustrated (FIG. 14 bottom). Here, too, it becomes clear that patients with a C-allele develop a relapse earlier than patients with the T-allele. In FIG. 15, the survival over time is illustrated. It can again be seen that patients with the GNA11 CC genotype die earlier than patients with the T-allele.

Various publications are referred to throughout this application. The disclosures of these publications are hereby incorporated by reference in their entireties into the subject application to more fully describe the art to which the subject application pertains.

What is claimed is:

1. An in vitro process for prognosis of disease risk, disease development, drug risk and/or for finding a drug target, the process comprising searching for one or more gene modification in the promoter region of the gene GNA11 and/or in intron 1 of the gene GNA11 on the human chromosome 19p13.3.

2. The process of claim 1, comprising searching for a polymorphism G(−659)C in a patient sample.

3. The process of claim 1, comprising searching for a polymorphism G(1606)T in a patient sample.

4. The process of claim 1, comprising searching for a polymorphism C(10564)T in a patient sample.

5. The process of claim 1, comprising searching for two or three of the polymorphisms promoter G(−659)C, intron 1G(1606)T and intron 1C(10564)T in a patient sample.

6. The process of claim 2, comprising searching for two or three of the polymorphisms promoter G(−659)C, intron 1G(1606)T and intron 1C(10564)T in a patient sample.

7. The process of claim 3, comprising searching for two or three of the polymorphisms promoter G(−659)C, intron 1G(1606)T and intron 1C(10564)T in a patient sample.

8. The process of claim 4, comprising searching for two or three of the polymorphisms promoter G(−659)C, intron 1G(1606)T and intron 1C(10564)T in a patient sample.

9. The process of claim 1, comprising searching for the polymorphism A(−761)C in a patient sample.

10. The process of claim 1, comprising searching for the polymorphism G(−626)A in a patient sample.

11. The process of claim 2, comprising searching for the polymorphism G(−626)A in a patient sample.

12. The process of claim 9, comprising searching for the polymorphism G(−626)A in a patient sample.
